(19) United States
(12) Patent Application Publication
(10) Pub. No.: US 2010/0241015 A1
(43) Pub. Date: Sep. 23, 2010

(54) OPTICAL SYSTEMS FOR DIAGNOSING AND MONITORING DERMAL MICROVASCULAR HEALTH

(75) Inventors: Danny Petrasek, Los Angeles, CA (US); Morteza Gharib, Altadena, CA (US); Alan H. Barr, Pasadena, CA (US); Eric M. Chin, Memphis, TN (US)

Correspondence Address: Milstein Zhang & Wu LLC, 49 Lexington Street, Suite 6, Newton, MA, 02465-1062, US

(73) Assignee: California Institute of Technology, Pasadena, CA (US)

(21) Appl. No.: 12/633,579

(22) Filed: Dec. 8, 2009

Related U.S. Application Data
(60) Provisional application No. 61/201,169, filed on Dec. 8, 2008.

(51) Int. Cl. A61B 5/02; G01N 21/55

(52) U.S. Cl. 600/504; 356/445

(57) ABSTRACT

The invention generally relates to a device for assessing dynamic microvascular refill (DMR), a novel measure of microvascular function. Microvascular refill is determined under dynamic conditions by monitoring changes in fingernail reflectance spectra in response to small shear forces applied to the fingernail. A hemodynamic model is described to examine the physiological significance of observed signals. The invention will provide healthcare workers with a simple, user friendly, non-invasive method of rapidly assessing microvascular function that would greatly facilitates the early detection and monitoring of the onset and treatment of vascular diseases.

### PRIORITY CLAIMS AND RELATED APPLICATIONS

This application claims the benefit of U.S. Provisional Application No. 61/201,169 filed Dec. 8, 2008, the entire content of which is expressly incorporated herein by reference.

### FIELD OF THE INVENTION

The invention relates to devices and methods for measuring microvascular health status of a subject. More particularly, the invention relates to devices and methods for measuring microvascular perfusion in a patient by monitoring a change in a subject's digit nail reflectance spectra in response to a force applied to the digit nail of the subject.

### BACKGROUND OF THE INVENTION

Atherosclerosis, the most prevalent of cardiovascular diseases, is the principal cause of death in the United States. The often-insidious onset of the disease results in the progressive formation of fibro-fatty and fibrous lesions or plaques within the blood vessel endothelium, preceded and accompanied by inflammation. When such vessels are occluded, various clinical syndromes may result from death of tissue previously nourished by the occluded vessels or inability of the vessels to transport sufficient blood supply to regions requiring high blood consumption and accompanying nutrients. In its most advanced stage, the sudden rupture of arteriosclerotic plaques can cause aortic occlusion resulting in heart attack or stroke and possible death.

Microcirculation, defined as blood flow through vessels averaging <0.3 mm diameter, is responsible for supplying blood to major organ systems of the body as well as the periphery such as the skin. The degree of blood perfusion in the cutaneous microvascular bed can provide a good indicator of peripheral vascular disease and can be indicative of the overall health of the vascular system. Patients with completely normal blood pressure can have severe impairment of microvascular circulation which is often an early symptom of otherwise undetected systemic disease.

Several disease states are known to affect microcirculation. Systemic diseases such as arthrosclerosis, diabetes mellitus, collagen vascular diseases, systemic hypertension, and chronic renal failure as well as arteriopathies such as Takayasu's Arteritis and Moyamoya disease can result in disorders of microvascular function. In addition, microvascular perfusion is a gauge for any skin injury or pathology ranging from burns, abrasions, pathological skin conditions such as psoriasis and others.

Current diagnostic methods for measuring microvascular perfusion include direct capillary pressure measurement; transcutaneous oxygen measurement; radionuclide techniques; temperature techniques (radiometric measurements, thermography, microwave radiometry, thermal clearance or conductivity measurements); ultrasound; dermofluorometry; laser Doppler flowmetry; and capillary microscopy. All of the above methods have been tried but are not in common use among general physicians and reside in the realm of radiology, vascular surgery, dermatology or other subspecialties.

There is therefore an unmet need in healthcare for a simple, user friendly, non-invasive method of rapidly assessing microvascular function that would facilitate the early detection and monitoring of the onset and treatment of vascular diseases.

### SUMMARY OF THE INVENTION

The invention is based in part on the unexpected discovery that much more effective and rapid assessment of a patient's microvascular functions can be achieved through a novel dynamic microvascular refill (DMR) technology platform. Microvascular refill is determined under dynamic conditions by monitoring changes in fingernail reflectance spectra in response to small shear forces applied to the fingernail. A hemodynamic model is described to examine the physiological significance of observed signals. The invention will provide healthcare workers with a simple, user friendly, non-invasive method of rapidly assessing microvascular function that would greatly facilitates the early detection and monitoring of the onset and treatment of vascular diseases.

In one aspect, the invention generally relates to a method for measuring a microvascular function of a subject. The method includes dynamically monitoring a change in a subject's digit nail reflectance spectra in response to a force applied to the digit nail of the subject, wherein the change corresponds to the microvascular refill of blood upon relaxation of the force. The digit nail may be a fingernail, e.g., an index fingernail.

In some embodiments, the reflectance spectrum is a full-spectrum reflectance spectrum. In some other embodiments, the reflectance spectrum ranges from about 300 nm to about 1,000 nm. In some preferred embodiments, the force is a shear force applied to the tip of the digit nail, where the force causes substantial blanching of the digit nail. In some embodiments, monitoring the change in a subject's digit nail reflectance spectra includes measuring the rate of microvascular refill.

In another aspect, the invention generally relates to a method for measuring a microvascular perfusion status of a subject. The method includes: applying a force to a digit nail of the subject, wherein the force is substantially parallel to the digit nail plate thereby causing blanching of at least a portion of the digit nail; and spectroscopically measuring blood refill to the blanched fingertip thereby measuring a microvascular perfusion status of the subject.

In another aspect, the invention generally relates to a device for measuring a microvascular function of a subject. The device includes: a pressure regulator; a light source; an imaging detector capable of recording reflectance spectra; and a positioning component for securely placing a subject's digit in position with the pressure regulator, the light source and the imaging detector.

In some embodiments, the light source is a light emitting diode or laser. The positioning component may be configured to apply a force to the subject's digit that is substantially parallel to the digit nail plate the subject digit. In certain preferred embodiments, the positioning component is configured to apply a force, e.g., a shear force, to the tip of the subject's digit.

In some embodiments, the imaging detector is capable of recording reflectance spectra ranging from about 350 nm to about 1,000 nm.

### BRIEF DESCRIPTION OF THE DRAWINGS

FIG. 1 shows exemplary blanching patterns. Compared to unblanched (a), nearly all of the nail blanches uniformly under lateral force (b) while fingertip compression produces a blanching at the fingertip and reddening at the base of the fingernail (c).

FIG. 2 shows an exemplary depiction of the vascular anatomy of the fingertip.

FIG. 3 shows an exemplary depiction of (a) Hemodynamic model, and b) electrical circuit representation of model.

FIG. 4 shows an exemplary schematic diagram of an embodiment of the invention.

FIG. 5 shows an exemplary schematic diagram of an embodiment of the invention.

FIGS. 6A and 6B shows exemplary depictions of an embodiment of the invention in measuring a subject's dermal microvascular perfusion.

FIG. 7 shows an exemplary depiction of: (a) Average spectrum with and without applied pressure; (b) Difference spectrum, shown with hemoglobin and oxyhemoglobin absorption spectra and (c) Magnitude of difference.

FIG. 8 shows an exemplary average response curves compared with applied pressure.

FIG. 9 shows an exemplary response curves for individual subjects showing (a) exponential and (b) sigmoid responses (average response is shown in bold).

### DETAILED DESCRIPTION OF THE INVENTION

Oxygen, which is critical to the survival of tissue, is carried to various parts of the body by the blood (vascular) system. The capillary nail refill test, historically part of the Triage Index, is a quick test that measures how well the vascular system is functioning in the extremities (hands and feet), which are the parts of the body farthest from the heart. The test typically involves squeezing the patient's fingernail pad, at a direction normal to the fingernail, pad to cause blanching under the nail and visually measuring the time it takes to restore color to the nail. If the subject is dehydrated, or tissue perfusion is blocked by other means, this quick test can alert a health care provider that care needs to be taken to restore normal vascular flow. The capillary nail refill test while useful in emergency situations is of limited use in clinical diagnosis because of poor reproducibility, specificity and sensitivity.

Although only semi-quantitative, the capillary refill test indicates that (i) that mechanical compression of the fingertip empties nailbed capillaries of blood, and (ii) that a measurable optical signal exists that corresponds to microvascular flow.

The invention seeks to significantly improve on the capillary refill test by modeling novel parameters to rapidly and accurately assess microvascular characteristics, in particular, maximum flow rate and microvascular elastic properties (such as compliance and the spring constant under a linear stress-strain model).

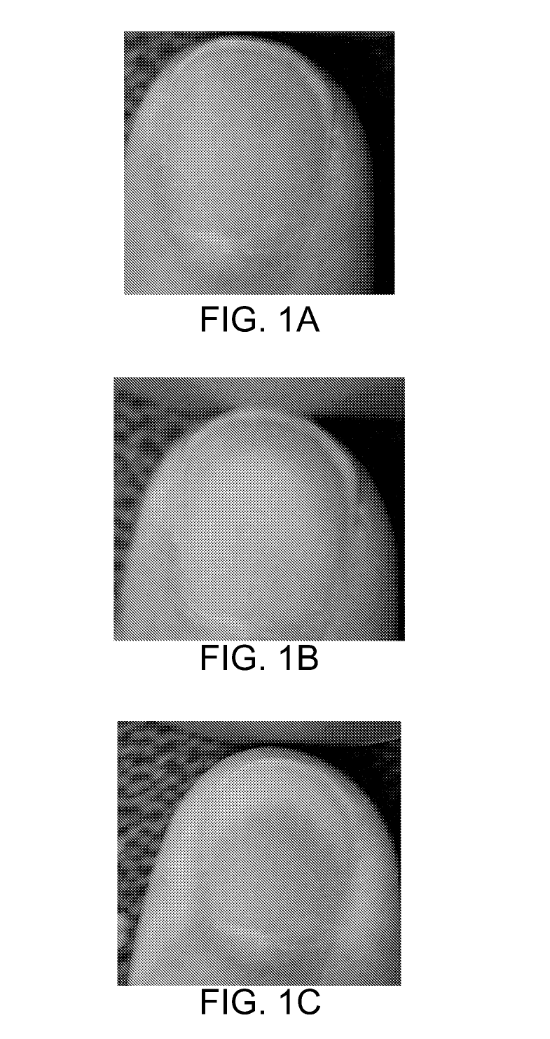

In an exemplary embodiment, the invention uses fingernail reflectance spectra measurements to evaluate microvascular function in the vascular bed under the fingernail. Full-spectrum reflectance is measured to determine blood-caused reflectance change under conditions where a shear force is applied to the tip of the fingernail parallel to the fingernail (as opposed to applying force normal to the finger plate) and then released. This induces a more uniform blanching over the surface of the fingernail (see FIG. 1) and allows a more accurate and reproducible measurement of the microvascular refill rate. A person of ordinary skill in the art will appreciate that this simple experimental approach is not limited just to finger nails but it can be applied in principle to any digit of a patient, such as a thumb nail, index fingernail or toe nail.

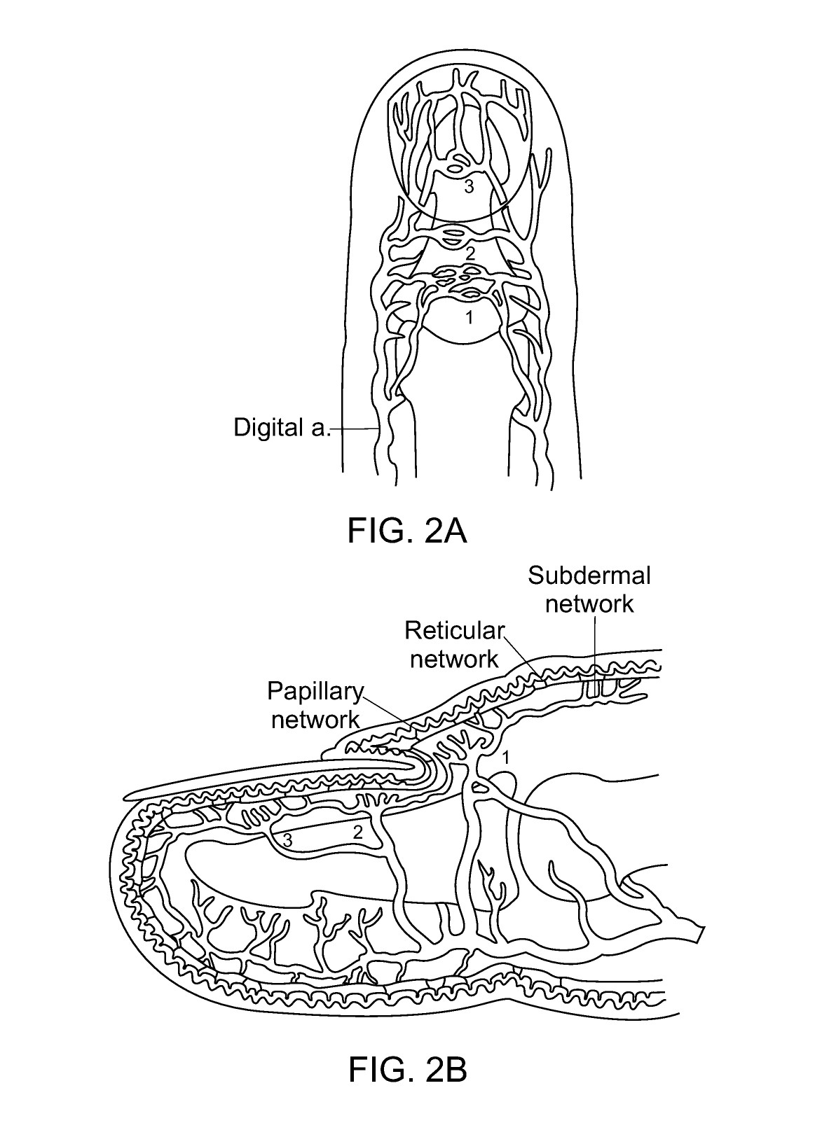

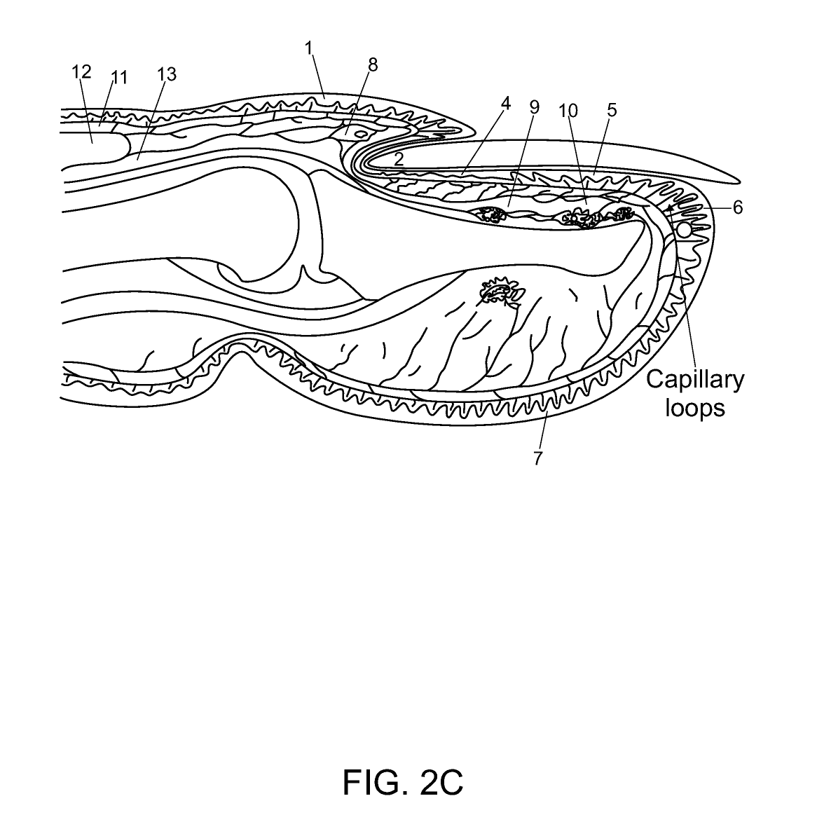

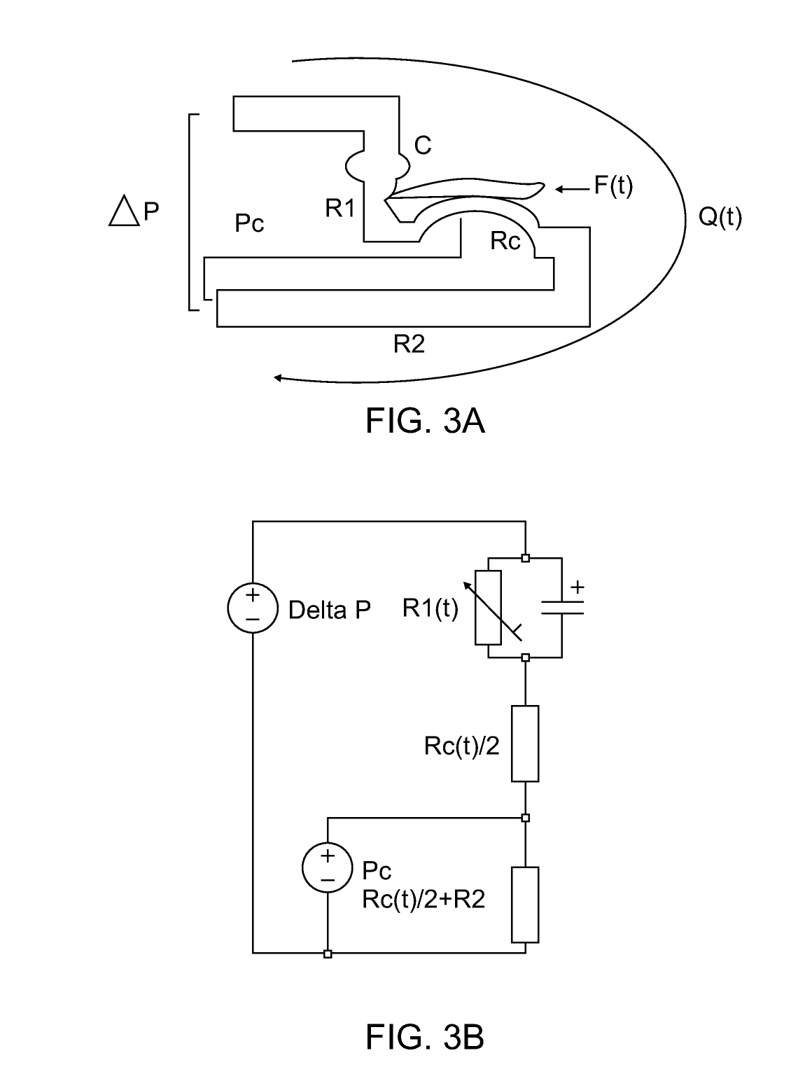

As shown in FIG. 2, the nailbed capillary system is fed dorsally from behind the nail plate by tributaries of the common palmar digital artery. By applying a shear force that drives the nail plate inwards, these tributaries are more easily occluded which in turn facilitates a more uniform blanching the nail. This is supported by the observation that on release of pressure, reddening proceeds from the proximal end of the nail.

### A Novel Hemodynamical Model for Dermal Microvascular Perfusion

Previous studies have measured fingernail coloration in response to various amounts of fingerpad pressure for the purpose of building a device that optically measures force applied to a subject's finger. (Mascaro, Stephen A. “Photoplethysmograph Fingernail Sensors for Measuring Finger Forces Without Haptic Obstruction.” IEEE Transactions on Robotics and Animation, Vol. 17, NO. 5, 2001.) Reflectance was measured through the fingernail at 800 nm (the isobestic point at which hemoglobin and oxyhemoglobin have identical absorption characteristic) in order to infer pressure applied by the subject's finger. In so doing, Mascaro et al. built a hemodynamic model of the fingertip in which capillaries and veins supply varying vascular resistance contributions and the effect of applied pressure is modeled by treating the vessel walls as damped springs.

Here, the invention substantially differentiates from this hemodynamical model in at least the following significant aspects:)

(1) Only the nailbed-supplying arteries are compressed by applied pressure. The effect of the compression is modeled as a single damped-spring unit (as opposed to the three damped-spring units reported in Marasco et al. that varies vascular resistance upstream of the capillary bed).

(2) When supplying arteries are occluded (increasing arterial resistance), flow is diverted to compliance effects upstream of the blockage. Reduced flow through the capillaries is the proposed mechanism for capillary blood volume reduction (assuming laminar flow).

### Mathematical Formulation

An applied pressure F(t) on the four damped-spring supplying arteries produces a displacement according to the 2nd-order ODE:

mΔ\acute{x}(t)=−kΔx(t)−bΔ\dot{x}(t)−−F(t)

Precapillary vascular resistance varies in response to the change in effective arterial diameter Δx(t) (assuming laminar flow is preserved) according to the Hagen-Poiseuille equation:

$${R}_{1} ( t ) = \frac{128 {L}_{A} {μ}_{blood}}{4 {π ( {D}_{A} + Δ x ( t ) )}^{4}}$$

Assuming that postcapillary resistance R2 and total pressure drop ΔP across the capillary system are constant, that pressure applied by interstitial fluid keeps intracapillary pressure constant at PC, and that capillary resistance RC(t) is uniformly distributed over the length of the capillary, the average capillary pressure is given by:

P_{c}=Q(t)[R₂+R_{c}(t)/2

Pressure drop P1(t) across the occluded supply arteries is given by:

P₁(t)=Q₁(t)R₁(t)

with continuing flow (i.e., flow not associated with the capacitative effect of the artery)

Q₁(t)=Q(t)−C\dot{P}₁(t)

and a total flow

$$Q ( t ) = \frac{2 ( Δ P - {P}_{1} ( t ) - {P}_{C} )}{{R}_{c} ( t )}$$

A second use of the Hagen-Poiseuille equation gives capillary volume as.

$$V ( t ) = \frac{{NL}_{C} π {{D}_{c} ( t )}^{2}}{4} = \sqrt{\frac{8 π {N}_{C} {L}_{c}^{3} {μ}_{blood}}{{R}_{C} ( t )}}$$

Optical signal is given by the Beer-Lambert Law

I(t)=I₀+I₁e^{-dV(t)}/V_{Tissue}

### Exemplary Physiological Parameters

The following physiological parameters were used to test dermal microvascular perfusion:

| | Explanation | Value used | |
|---|---|---|---|
| m | arterial wall and blood inertia | ~10⁻³ | kg |
| k | arterial wall stiffness | ~10³ | N/m |
| b | arterial wall damping | ~10² | N · s/m |
| L_{A} | length of induced resistance vessel | ~10⁻³ | m |
| μ_{blood} | blood viscosity | ~3 × 10−3 | Pa · s |
| D_{A} | nominal supply artery diameter | ~10⁻¹ | m |
| R₂ | lumped venule resistance | ~10¹¹ | Pa · s/m³ |
| C | arterial compliance | | |
| ΔP | total pressure gradient | ~10⁴ | Pa |
| N | number of nailbed capillaries | ~3500 | |
| D_{C} | nominal capillary diameter | ~10⁻³ | m |
| L_{C} | average length of capillaries | ~10⁻³ | m |

### Measuring Dermal Microvascular Perfusion

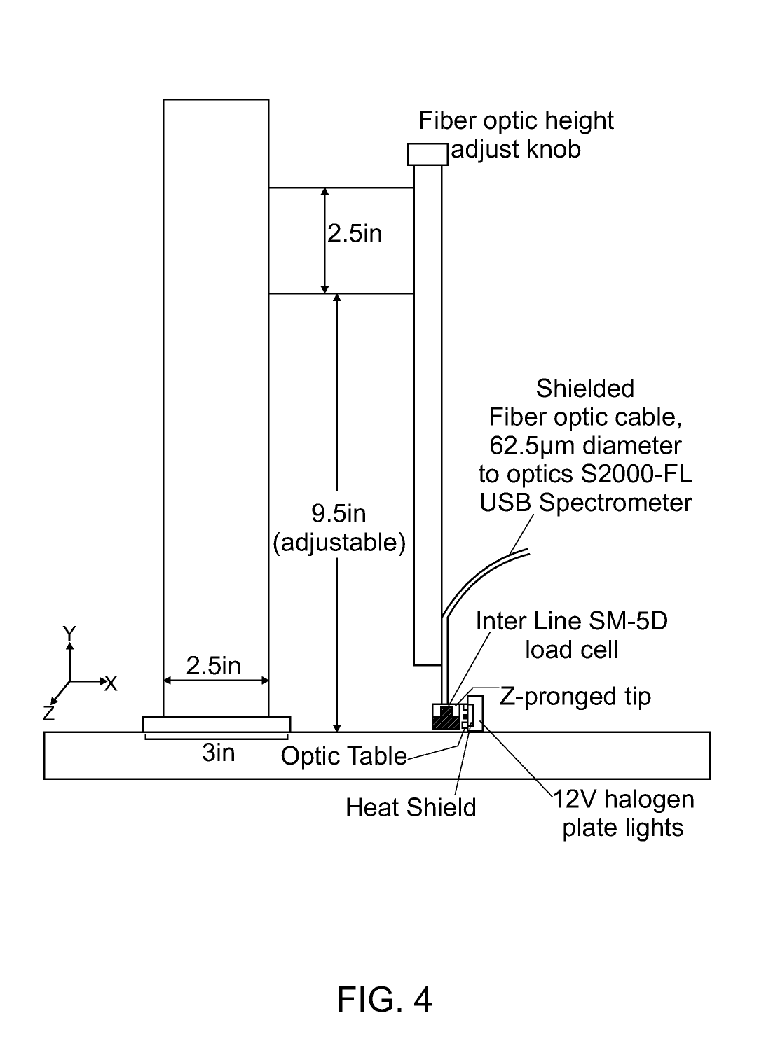

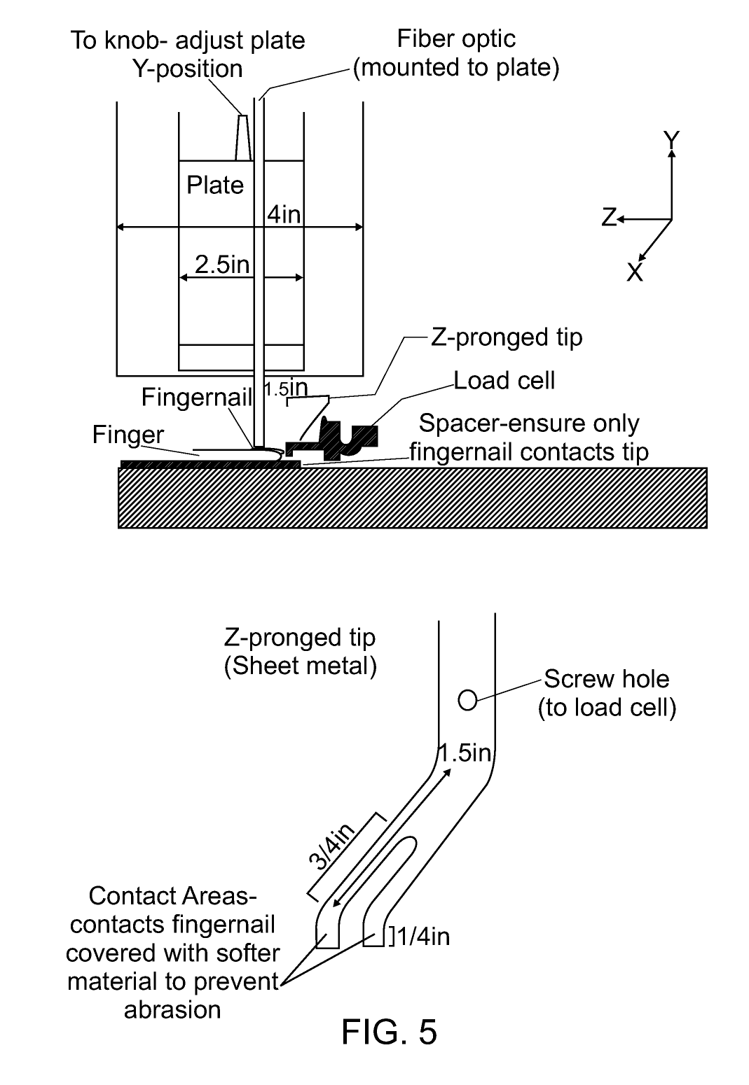

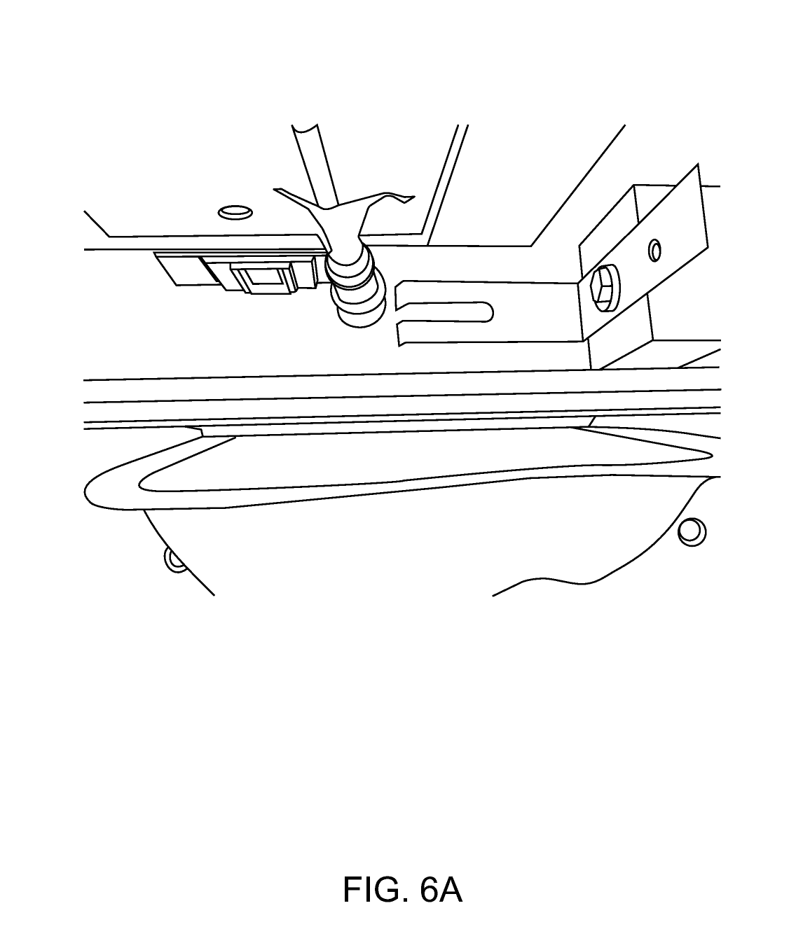

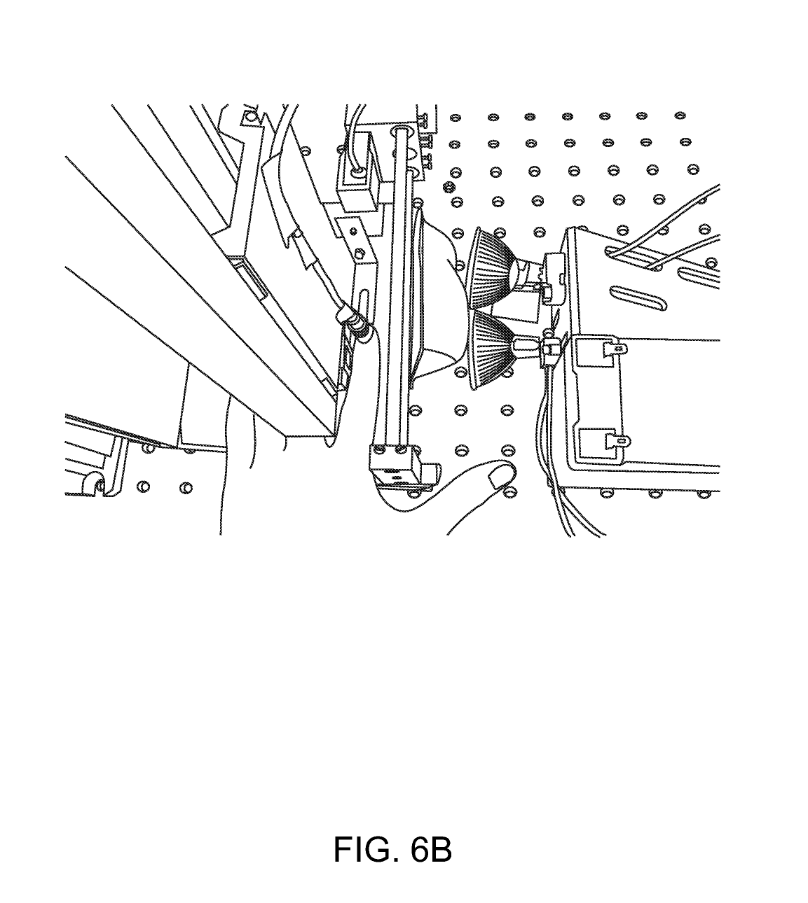

FIGS. 4, 5 and 6A and 6B show exemplary embodiments of an apparatus of the invention for measuring dermal microvascular perfusion is shown. Subjects apply force from the front of their fingernails to the load cell via a flat, metal, two-pronged applicator mounted to an Interface SM-50 load cell. Subjects are shown real-time force readings from the load cell and self-modulate the applied force. The force applied to the tip of fingernail can vary from a force sufficient to cause complete blanching at the tip of the finger as can be seen in FIG. 1 to a force that causes the initial onset of blanching. Complete blanching is defined as the force beyond which no additional blanching can be achieved. Exemplary forces for testing will dependent on the digit tested, for example toenail or fingernail, the length of the nail and the angle of the applied force with respect to the plane formed by the nail surface. Microvascular refill under the tip of a fingernail can be assessed from about 1%, 2%, 5%, 10%, 20%, 30%, 40%, 50%, 60%, 70%, 80%, 90%, 99% or 100% of the force required for complete blanching.

Within the same plane as the two-pronged applicator, a positioning component guides the location of the tip of a subject's fingernail for optimal interaction with the applicator, the light source and the imaging detector. To permit reproducible and accurate readings, an optional strap may be used to secure the finger to the positioning component. In certain embodiments, the bottom of the positioning component may be attached to an adjustable rail in a manner that allows the positioning components to move along the plane formed by the rail. Actuators permit the operator to change the angle the rail with respect to the plane formed by the two prongs of the applicator. In other embodiments, the position and angle of the fingernail tip can be assessed by a computer means and appropriate software. Hence, repeated measurements of dermal microvascular perfusion can be performed over months or years using the same parameters such as force applied and the angle of fingertip with the respect to the plane formed by the two-pronged applicator. In other embodiments, microvascular perfusion in the fingertip of a patient can be determined at different temperatures to determine vascular tone. For example, perfusion can be measured after immersion of the test finger in iced water for variable lengths of time ranging from 1 to 20 minutes, e.g., 2, 3, 4, 5, 6, 7, 8, 9, 10, 12, 15, 20 minutes.

Full-spectrum (λ=350-1000 nm) visible/NIR reflectance is acquired using an S2000-FL fiber optic spectrum analyzer (Ocean Optics, Inc.). The tip of the fiber optic is fixed 0.005 in above center of fingernail. Illumination is provided by one 12V, 50W halogen flood bulb.

Subjects are instructed to apply a stepwise constant force. Fingernail reflectance spectrum is measured for different applied forces. For example, one protocol requires (1) applying no force to the load cell for 10 seconds, then (2) applying 0.2 lb of force to the load cell for 10 seconds; and this continues for 5 minutes.

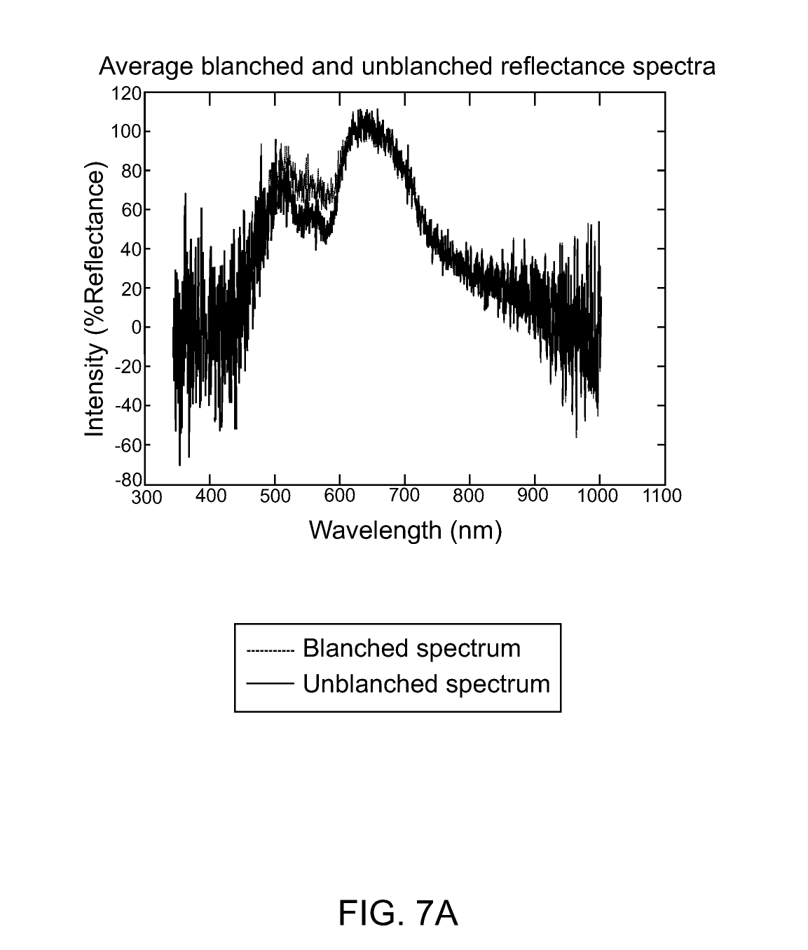

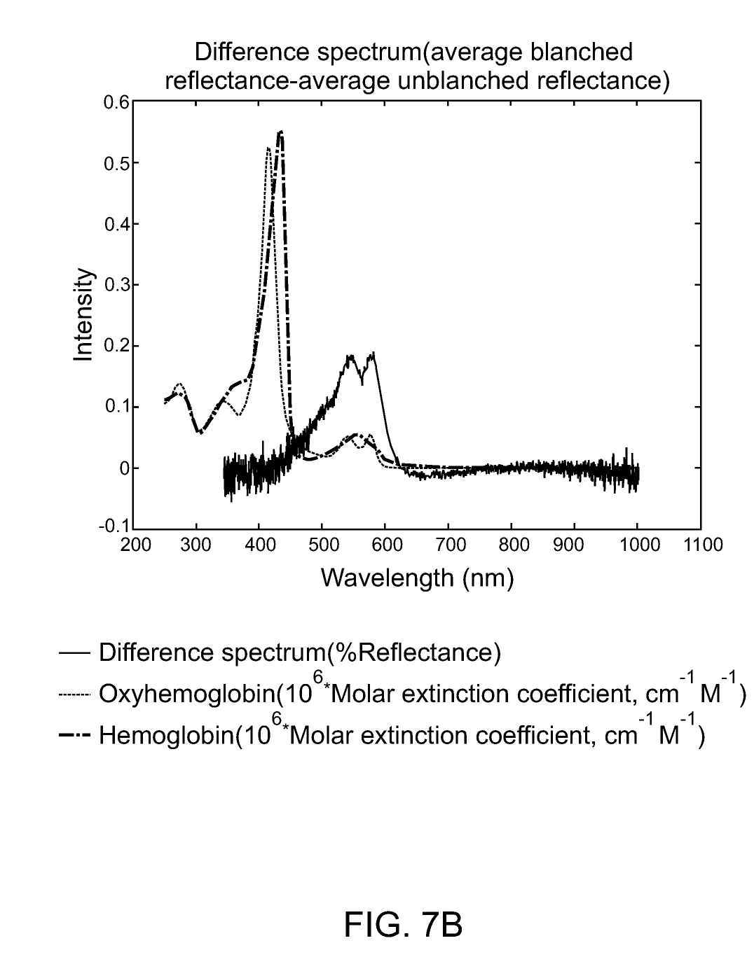

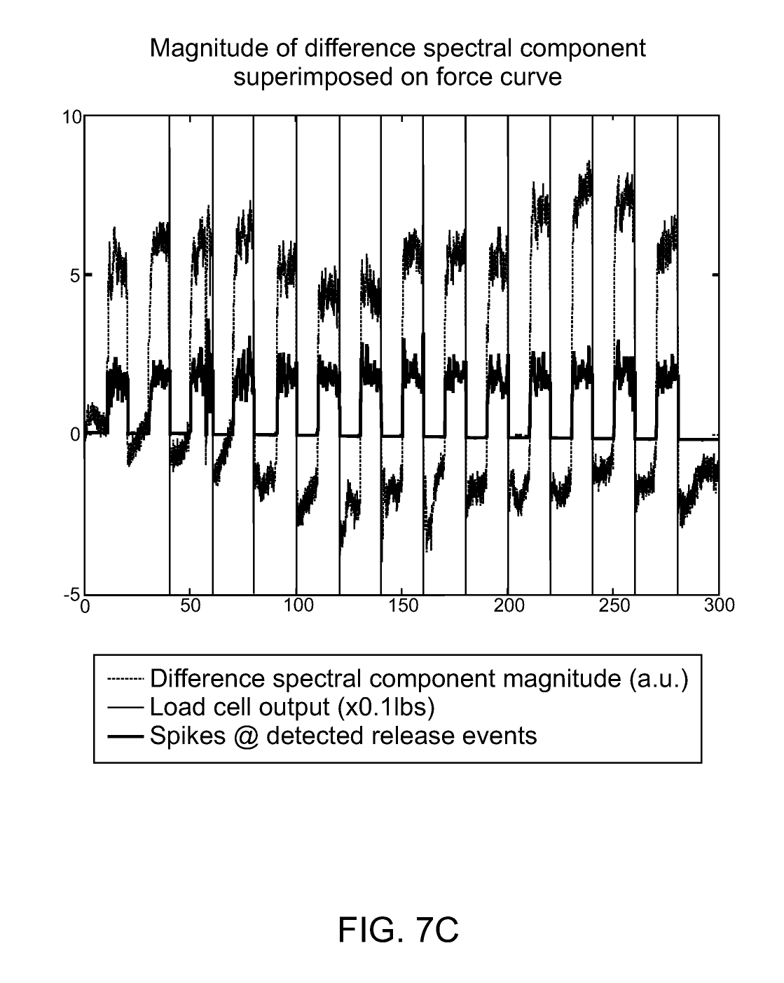

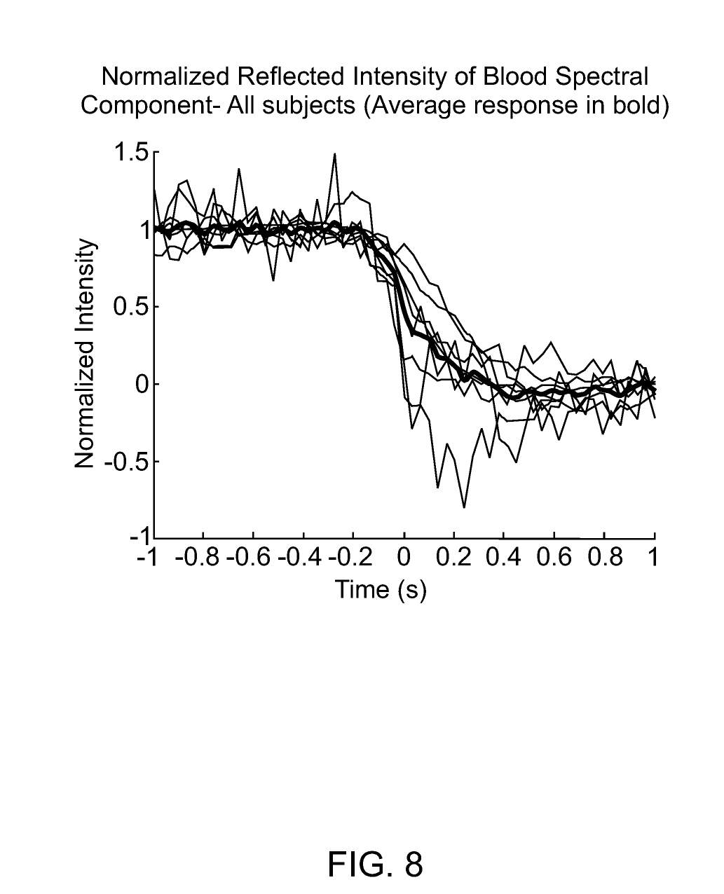

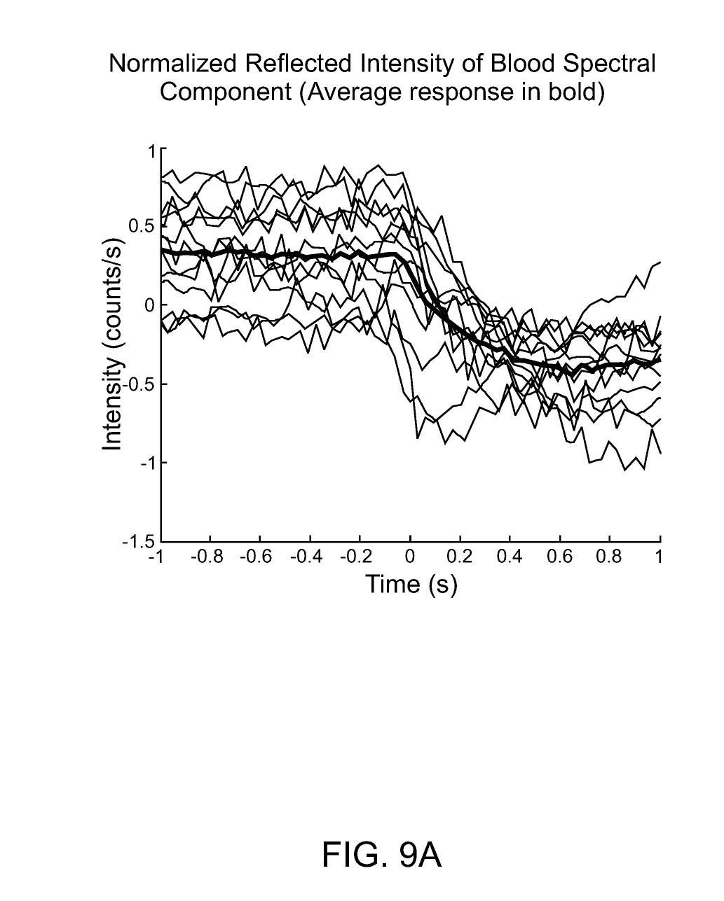

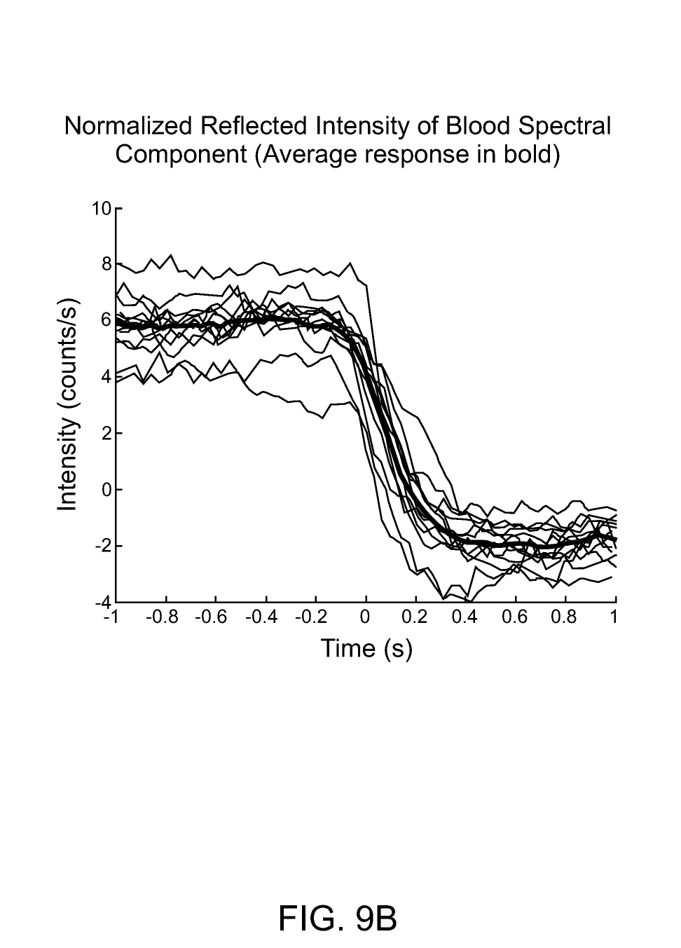

Microvascular perfusion was tested on 9 healthy, normal volunteers using this protocol. The time-varying component of the observed spectra was isolated for each subject and tracked the time-course of the magnitude of this component (see FIG. 7). Events were identified at which pressure was released and the responses of the magnitude of the time-varying spectral component over such events were compiled. Response curves varied from a sharp, exponential shape to a smooth sigmoid shape (see FIG. 8 and FIG. 9). In FIG. 9, the difference signal in one subject did not have the characteristic oxyhemoglobin double peak (data for this subject excluded). Signal to noise was significantly higher when tracking the first principal component of variation (rather than the blanched/unblanched difference signal) for two additional subjects.

While the presence of prominent oxyhemoglobin peaks in difference spectra indicates that blood flow is responsible for a significant portion of the observed signal, respective contributions of blood and interstitial fluid can be ascertained using electrical impedance plethysmography.

Varying the amplitude of applied pressure may also yield additional diagnostic information. As saturation behavior has been anecdotally observed (no further observable blanching occurs beyond a threshold pressure), steady-state measurements reflecting mechanical properties of small arteries may be obtained.

Thus, in one aspect, the invention generally relates to a method for measuring a microvascular function of a subject. The method includes dynamically monitoring a change in a subject's digit nail reflectance spectra in response to a force applied to the digit nail of the subject, wherein the change corresponds to the microvascular refill of blood upon relaxation of the force. The digit nail may be a fingernail, e.g., an index fingernail.

In some embodiments, the reflectance spectrum is a full-spectrum reflectance spectrum. In some other embodiments, the reflectance spectrum ranges from about 350 nm to about 1,000 nm. In some preferred embodiments, the force is a shear force applied to the tip of the digit nail, where the force causes substantial blanching of the digit nail. In some embodiments, monitoring the change in a subject's digit nail reflectance spectra includes measuring the rate of microvascular refill.

In another aspect, the invention generally relates to a method for measuring a microvascular perfusion status of a subject. The method includes: applying a force to a digit nail of the subject, wherein the force is substantially parallel to the digit nail plate thereby causing blanching of at least a portion of the digit nail; and spectroscopically measuring blood refill to the blanched fingertip thereby measuring a microvascular perfusion status of the subject.

In another aspect, the invention generally relates to a device for measuring a microvascular function of a subject. The device includes: a pressure regulator; a light source; an imaging detector capable of recording reflectance spectra; and a positioning component for securely placing a subject's digit in position with the pressure regulator, the light source and the imaging detector.

In some embodiments, the light source is a light emitting diode or laser. The positioning component may be configured to apply a force to the subject's digit that is substantially parallel to the digit nail plate the subject digit. In certain preferred embodiments, the positioning component is configured to apply a force, e.g., a shear force, to the tip of the subject's digit.

In some embodiments, the imaging detector is capable of recording reflectance spectra ranging from about 300 nm to about 1,000 nm, from about 350 nm to about 900 nm, from about 400 nm to about 800 nm, for example.

In other embodiments, the microvascular perfusion test can be assessed in pools of subjects with and without diagnosed microvasculature-compromising diseases and the results of the test could be correlated with other diagnostic indicators of cardiovascular disease such as blood pressure, reactive C-protein, triglyceride and cholesterol levels.

In alternative embodiments, cross-validation with more direct methods such as capillaroscopy could be performed.

### INCORPORATION BY REFERENCE

References and citations to other documents, such as patents, patent applications, patent publications, journals, books, papers, web contents, have been made in this disclosure. All such documents are hereby incorporated herein by reference in their entirety for all purposes.

### EQUIVALENTS

The representative examples which follow are intended to help illustrate the invention, and are not intended to, nor should they be construed to, limit the scope of the invention. Indeed, various modifications of the invention and many further embodiments thereof, in addition to those shown and described herein, will become apparent to those skilled in the art from the full contents of this document, including the examples which follow and the references to the scientific and patent literature cited herein. The following examples contain important additional information, exemplification and guidance which can be adapted to the practice of this invention in its various embodiments and equivalents thereof.

What is claimed is:

1. A method for measuring a microvascular function of a subject, comprising dynamically monitoring a change in a subject's digit nail reflectance spectra in response to a force applied to the digit nail of the subject, wherein the change corresponds to the microvascular refill of blood upon relaxation of the force.

2. The method of claim 1, wherein the digit nail is a fingernail.

3. The method of claim 2, wherein the fingernail is an index fingernail.

4. The method of claim 1, wherein the reflectance spectra is a full-spectrum reflectance spectra.

5. The method of claim 1, wherein the reflectance spectra ranges from about 350 nm to about 1,000 nm.

6. The method of claim 1, wherein the force is applied to the tip of the digit nail.

7. The method of claim 1, wherein the force is a shear force.

8. The method of claim 1, wherein the force causes substantial blanching of the digit nail.

9. The method of claim 1, wherein monitoring the change in a subject's digit nail reflectance spectra comprises measuring the rate of microvascular refill.

10. A method for measuring a microvascular perfusion status of a subject, comprising:
applying a force to a digit nail of the subject, wherein the force is substantially parallel to the digit nail plate thereby causing blanching of at least a portion of the digit nail; and
spectroscopically measuring blood refill to the blanched fingertip thereby measuring a microvascular perfusion status of the subject.

11. The method of claim 10, wherein the digit nail is a fingernail.

12. The method of claim 11, wherein the fingernail is an index fingernail.

13. The method of claim 10, wherein the spectroscopically measuring blood refill step comprises a full-spectrum reflectance spectrum.

14. The method of claim 10, wherein the force is applied to the tip of the digit nail.

15. The method of claim 10, wherein the force is a shear force.

16. The method of claim 10, wherein the force causes substantial blanching of the digit nail.

17. The method of claim 10, wherein measuring the microvascular perfusion status of a patient comprises measuring the rate of microvascular refill.

18. A device for measuring a microvascular function of a subject, comprising:
a pressure regulator;
a light source;
an imaging detector capable of recording reflectance spectra; and
a positioning component for securely placing a subject's digit in position with the pressure regulator, the light source and the imaging detector.

19. The device of claim 18, wherein the light source is a light emitting diode or laser.

20. The device of claim 18, wherein the positioning component is configured to apply a force to the subject's digit that is substantially parallel to the digit nail plate the subject digit.

21. The device of claim 18, wherein the positioning component is configured to apply a force to the tip of the subject's digit.

22. The device of claim 18, wherein the force is a shear force.

23. The device of claim 18, wherein the digit nail is a fingernail.

24. The device of claim 23, wherein the fingernail is an index fingernail.

25. The device of claim 18, wherein the imaging detector is capable of recording reflectance spectra ranging from about 350 nm to about 1,000 nm.
